(19) United States
(12) Patent Application Publication
(10) Pub. No.: US 2015/0238202 A1
(43) Pub. Date: Aug. 27, 2015

(54) LEG ALIGNMENT APPARATUS AND METHOD

(71) Applicant: CORIN LIMITED, Cirencester (GB)

(72) Inventors: Simon Collins, Tetbury (GB); Harsimran Sabharwal, Bristol (GB); Andrew Craig Sutcliffe, Nottingham (GB); Gianluca Girotti, Cheltenham (GB); Johan Bellemans, Langdorp (BE); Jan Maria Karel Victor, Brugge (BE); Ian James Elmslie, Cirencester (GB)

(21) Appl. No.: 14/433,040

(22) PCT Filed: Sep. 27, 2013
(86) PCT No.: PCT/GB2013/052528

§ 371 Date: Apr. 2, 2015

(30) Foreign Application Priority Data
Sep. 27, 2012 (GB) 1217679.8

(51) Int. Cl. A61B 17/15; A61B 17/02

(52) U.S. Cl. CPC A61B 17/155; A61B 17/025; A61B 2017/0268; A61B 2019/467

(57) ABSTRACT

Leg alignment apparatus for use in total arthroplasty of left and right knees, comprises an intramedullary rod, a femoral body for location on or adjacent to an anterior surface of a femur, and a support element which extends or is extendable from the femoral body to the intramedullary rod for holding the femoral body stationary or substantially stationary relative to the intramedullary rod. A condylar locator which is pivotable relative to the femoral body and which is seatable on a distal end of the femur is also provided, along with a soft-tissue tensioning element to apply a separation between the femur and the tibia, and a varus/valgus indicator to indicate an angular alignment of the knee based on the intramedullary rod and the soft-tissue tensioning element. A method of determining a distal end resection position for a femur at a knee joint is also provided.

The present invention relates to leg alignment apparatus for use in total arthroplasty of left and right knees, and to a method of determining a distal end resection position for a femur of a knee joint.

Currently, it is common surgical practice to bring a varus or valgus knee back to neutral alignment during total knee arthroplasty. This, however, may not always be the most appropriate course of action, and it has been found that some patients and even athletes may have improved performance with a varus knee or alignment other than neutral.

Additionally, it is common practice to resect both the tibia and the femur to set the knee alignment. This can result in soft-tissue and/or alignment imbalance that may require further surgical resection or ligament release.

The present invention seeks to provide a solution to these problems.

According to a first aspect of the invention, there is provided leg alignment apparatus for use in total arthroplasty of left and right knees, the apparatus comprising an intramedullary rod, a femoral body for location on or adjacent to an anterior surface of a femur, a support element which extends or is extendable from the femoral body to the intramedullary rod for holding the femoral body stationary or substantially stationary relative to the intramedullary rod, a condylar locator which is pivotable relative to the femoral body and which is seatable on a distal end of the femur, a soft-tissue tensioning element to apply a separation between the femur and the tibia, and a varus/valgus indicator to indicate an angular alignment of the knee based on the intramedullary rod and the soft-tissue tensioning element.

Preferable and/or optional features of the first aspect of the invention are set forth in claims 2 to 19, inclusive.

According to a second aspect of the invention, there is provided a method of determining a distal end resection position for a femur at a knee joint, the method comprising the steps of: a] prior to distal resection and with a leg in extension, providing a separation between a tibia and femur of the knee joint; b] with the leg still in extension and the separation of step a] applied, determining angular alignment of the knee based on an intramedullary rod extending along a femoral anatomical axis and/or a condylar plane and the separation between the tibia and femur; and c] determining a position of distal resection from a condylar plane of the femur.

Preferable and/or optional features of the second aspect of the invention are set forth in claims 21 to 34, inclusive.

The invention will now be more particularly described, by way of example only, with reference to the accompanying drawings, in which:

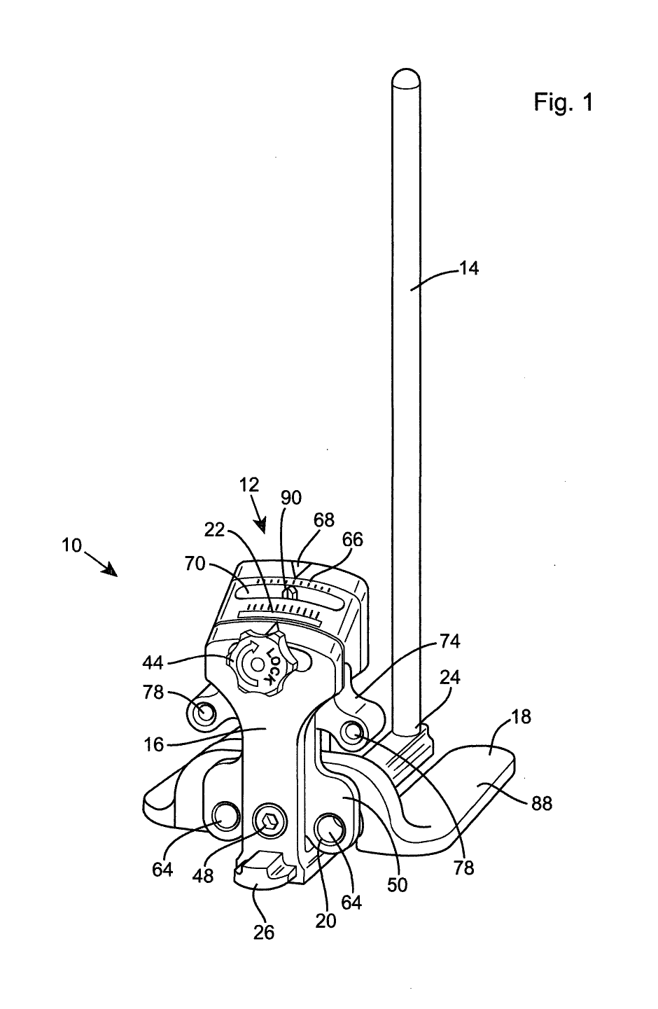

FIG. 1 shows a perspective view of a first part of one embodiment of leg alignment apparatus, in accordance with the first aspect of the invention;

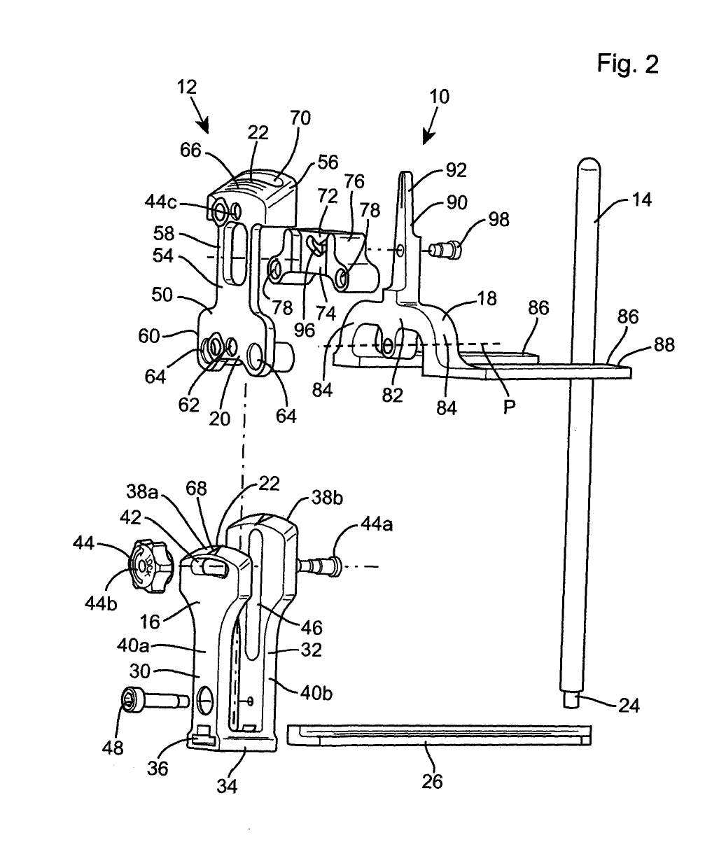

FIG. 2 shows an exploded view of the part of the leg alignment apparatus, shown in FIG. 1;

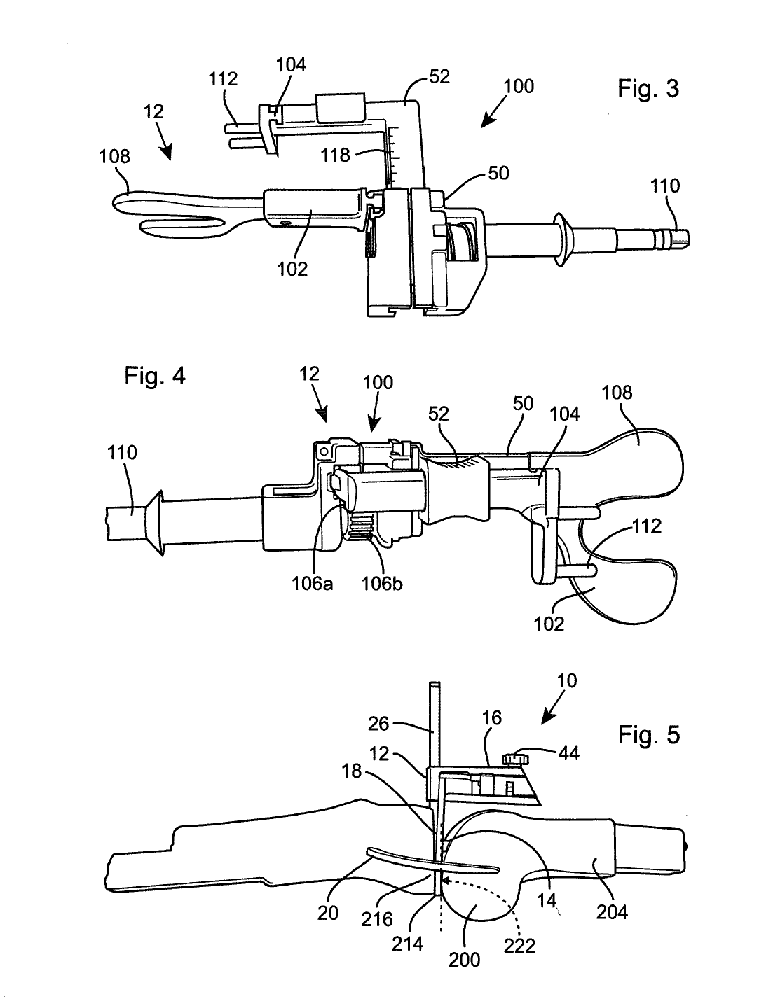

FIG. 3 is a side perspective view from below of a soft-tissue tensioner forming a second part of the embodiment of leg alignment apparatus, in accordance with the first aspect of the invention;

FIG. 4 is a top plan view of the soft-tissue tensioner, shown in FIG. 3;

FIG. 5 shows a side view of a knee joint with the first part of the leg alignment apparatus located thereon, the second part being omitted for clarity;

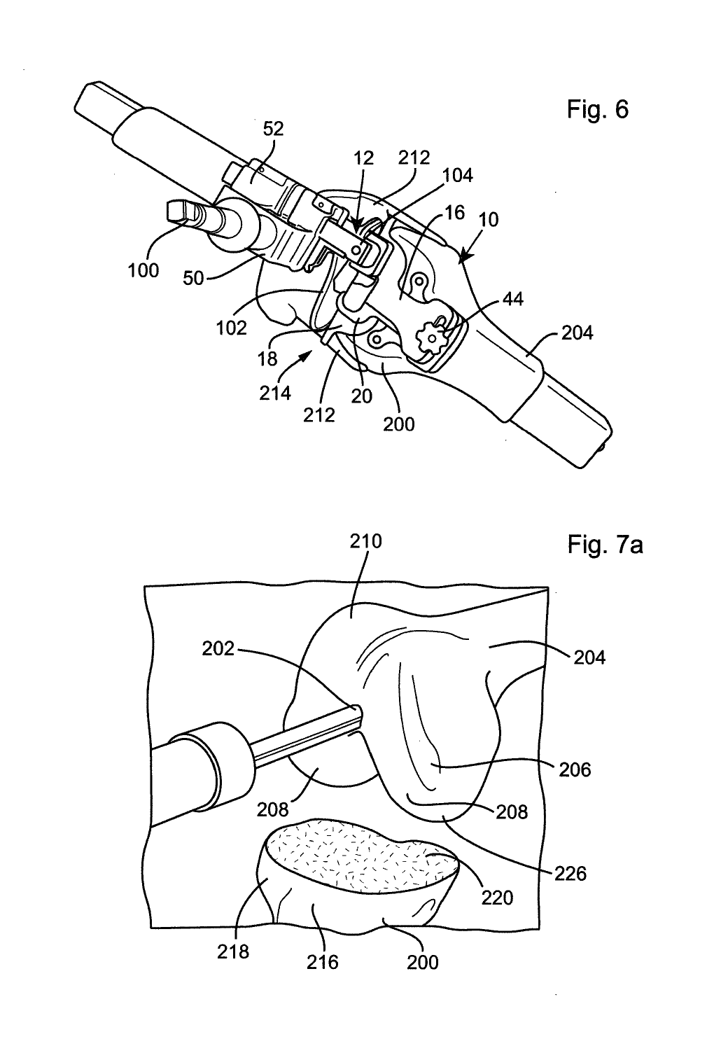

FIG. 6 shows an anterior plan of the knee joint with the first and second parts of the leg alignment apparatus located thereon;

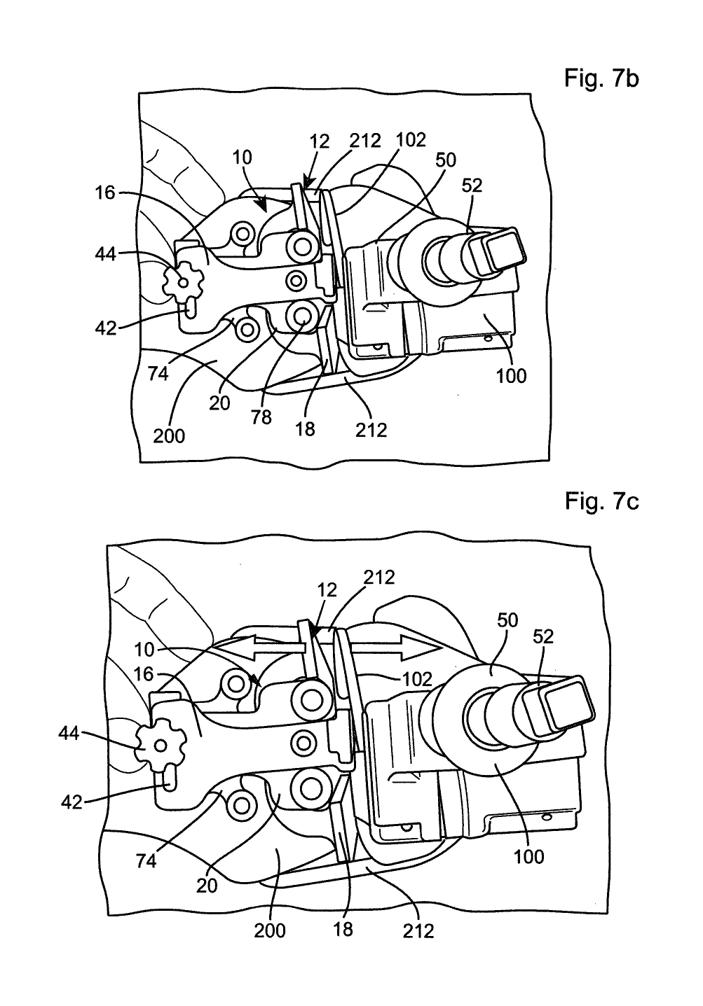

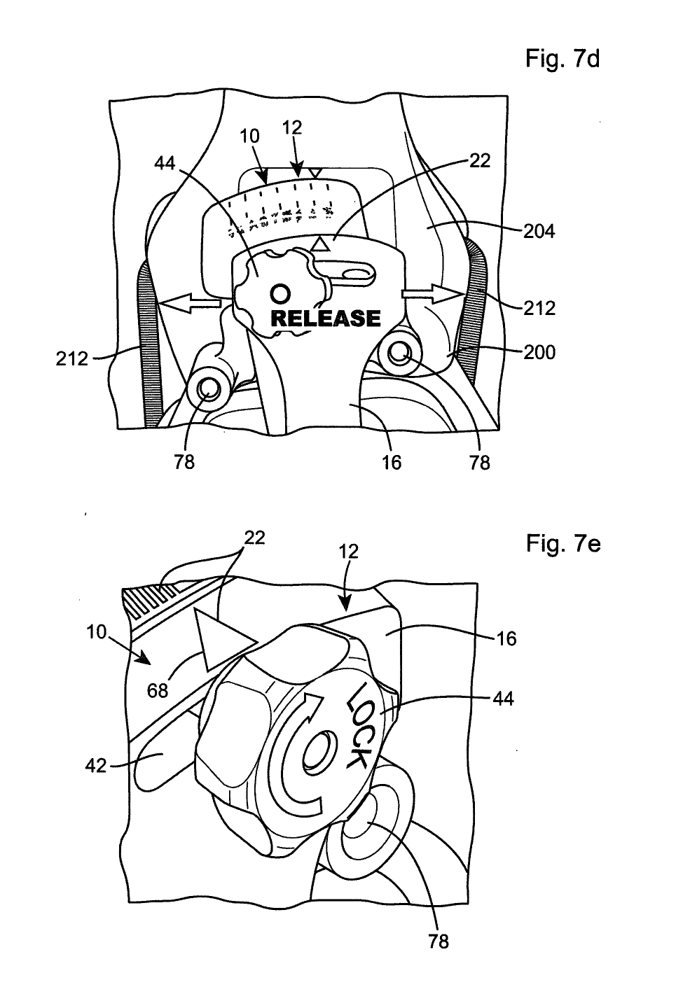

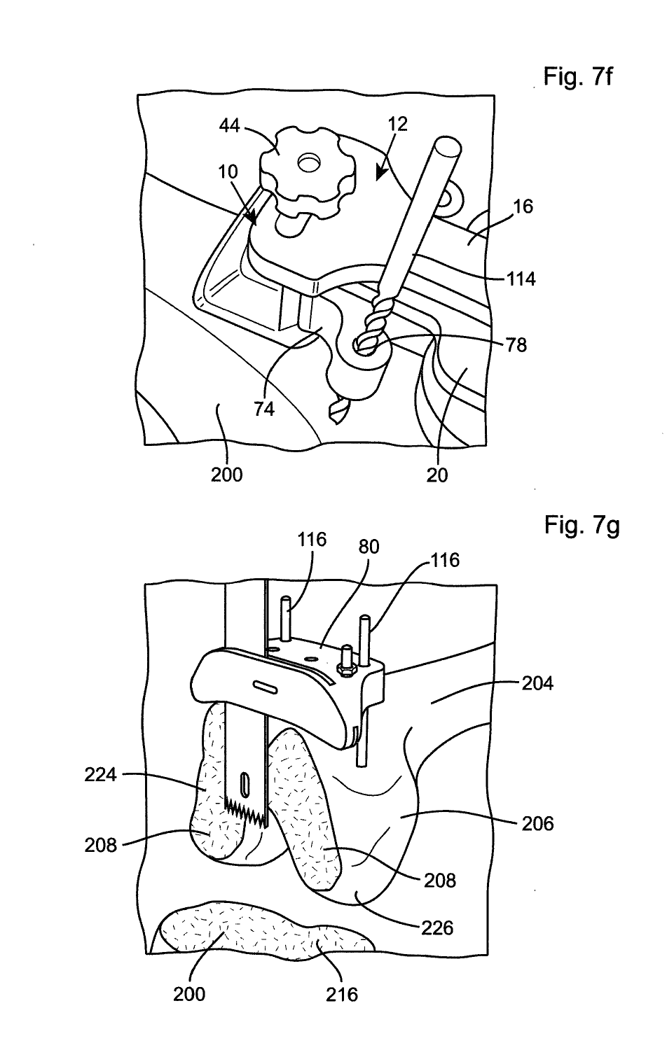

FIGS. 7a to 7g show a neutral alignment technique, using the leg alignment apparatus, and FIGS. 7b and 7c show a soft-tissue tensioning element with a connector engager removed so that the meniscal compartment can be more easily viewed;

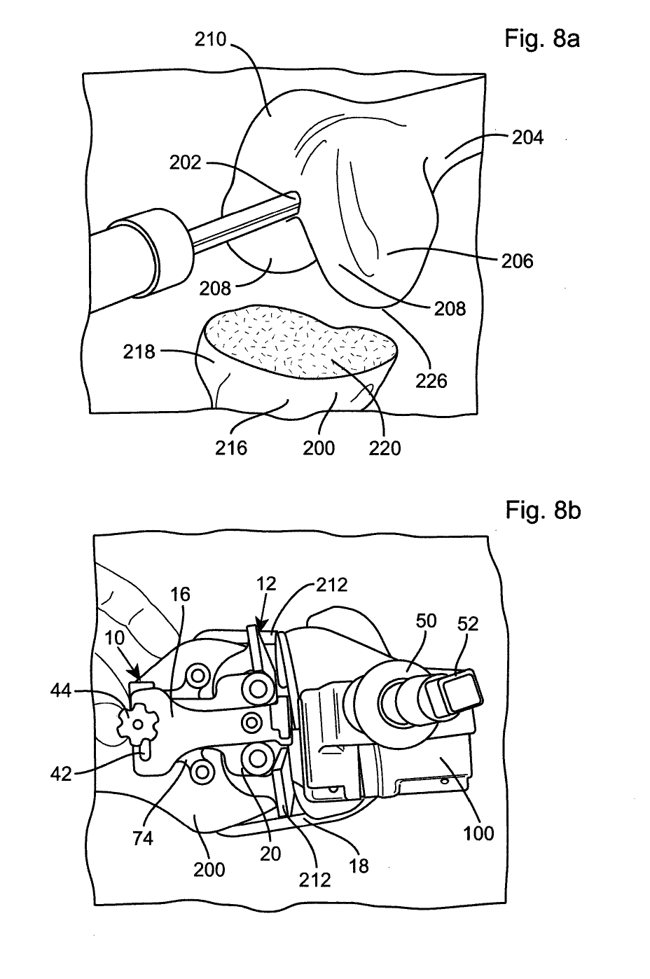

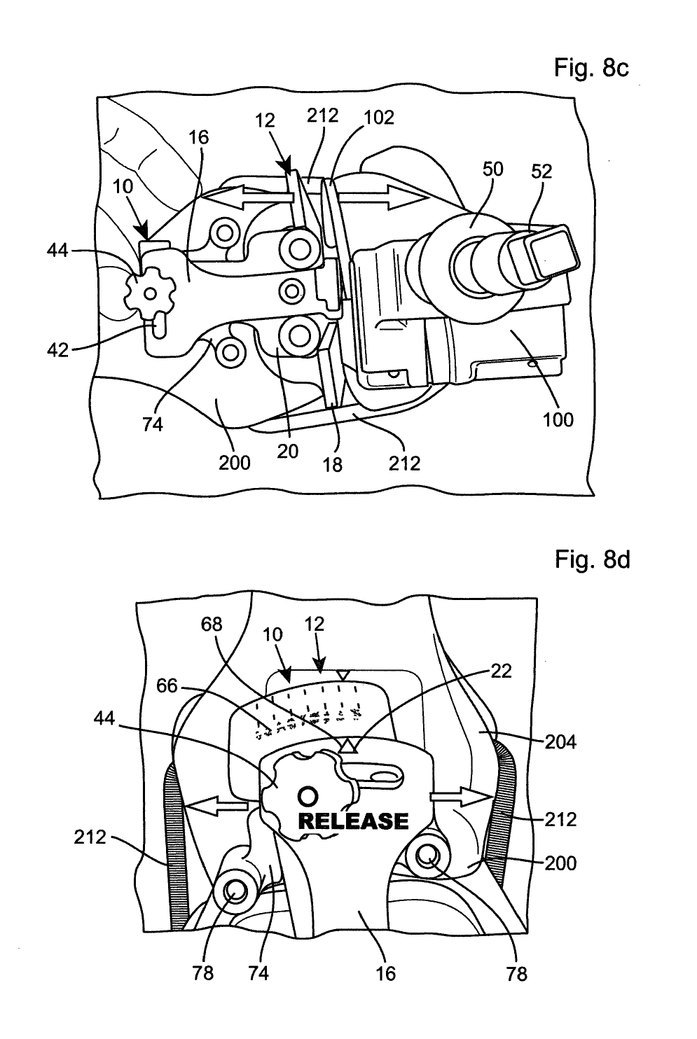

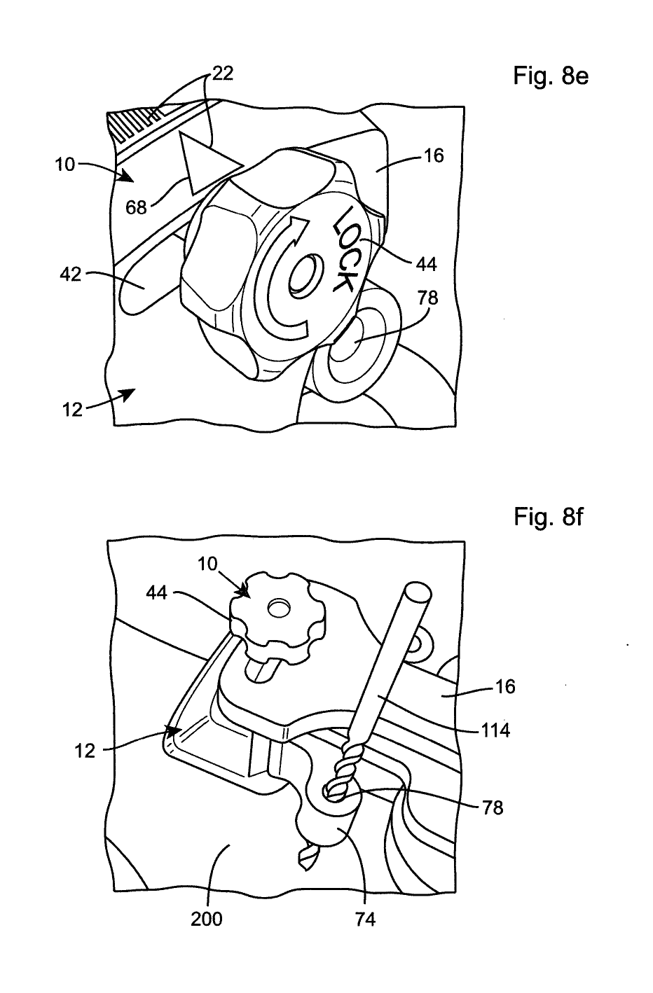

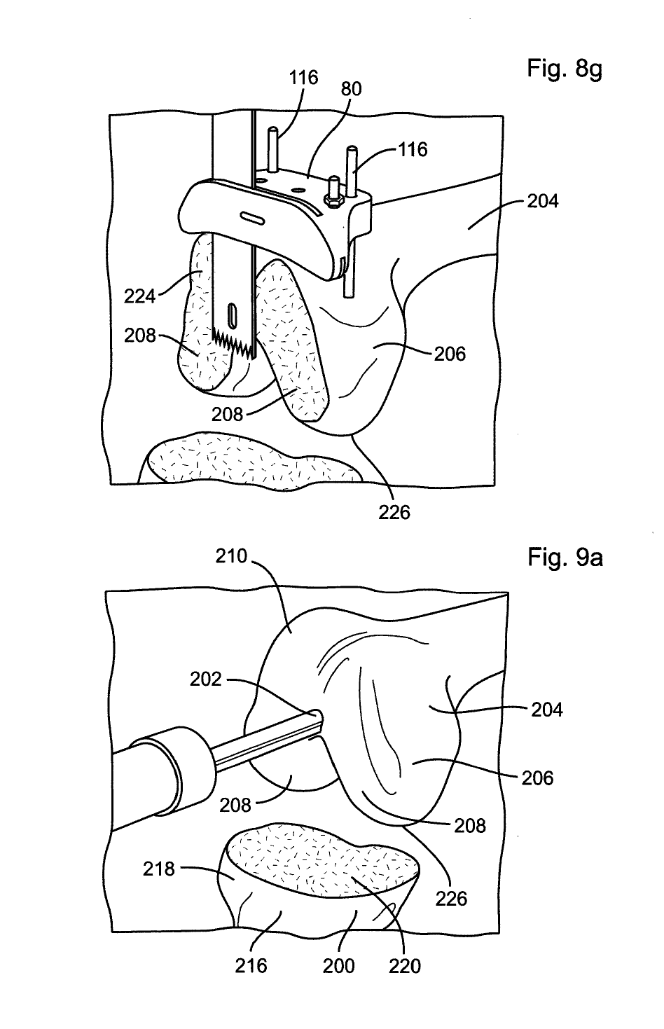

FIGS. 8a to 8g show a constitutional varus/valgus technique, using the leg alignment apparatus, and FIGS. 8b and 8c show a soft-tissue tensioning element with a connector engager removed so that the meniscal compartment can be more easily viewed; and

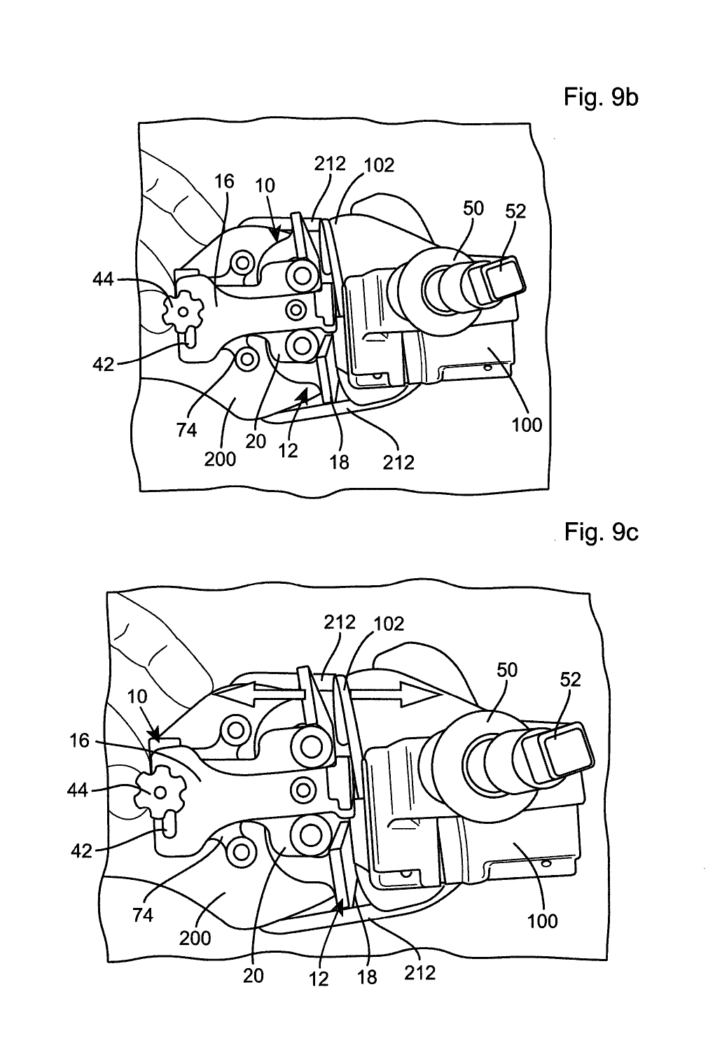

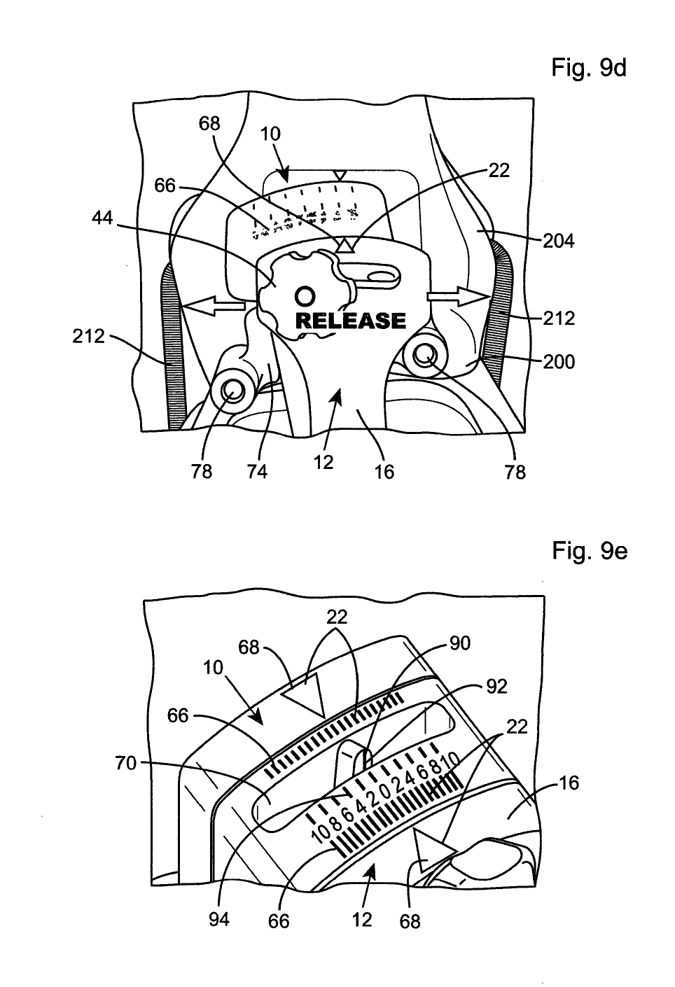

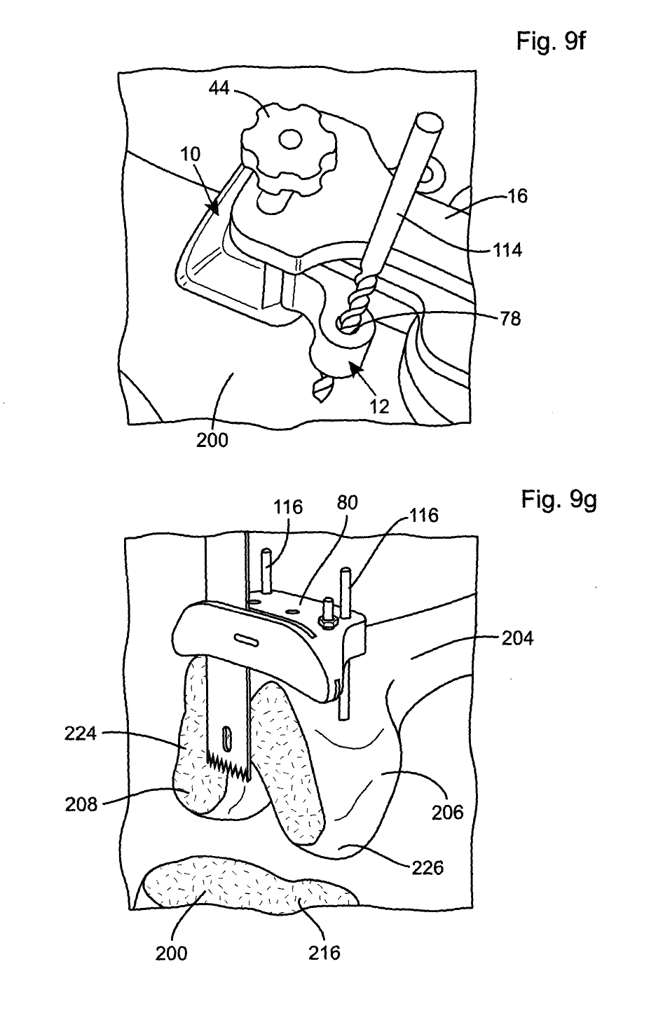

FIGS. 9a to 9g show a modified varus/valgus technique, using the leg alignment apparatus, and FIGS. 9b and 9c show a soft-tissue tensioning element with a connector engager removed so that the meniscal compartment can be more easily viewed.

Referring firstly to FIGS. 1 and 2 of the drawings, there is shown a first part 10 of an embodiment of leg alignment apparatus 12 for use in total arthroplasty of both left and right knees 200. The first part 10 comprises an intramedullary rod 14, femoral body 16, condylar locator 18, soft-tissue tensioning connector 20, and a varus/valgus indicator 22.

The intramedullary rod 14 is an elongate shaft-like element which, following reaming, is insertable into the intramedullary canal 202 of a patient's femur 204. The intramedullary rod 14, once inserted, aids in defining the anatomical axis of the femur 204. Reaming and insertion in this case takes place from the distal end 206 of the femur 204 and with the knee 200 in flexion.

The intramedullary rod 14 is rigid and preferably linear, and may conveniently be an already known device.

A distal end 24 of the intramedullary rod 14 which extends from the distal end 206 of the femur 204 is connected to the aforementioned femoral body 16 by a support element 26. In this case, the support element 26 is a separate plate which is engagable with the intramedullary rod 14 to extend at or substantially at right angles to a longitudinal axis of the intramedullary rod 14. The support element 26 has a lateral extent which is suitable for extending in an anterior direction between the medial and lateral distal condyles 208 of the femur 204. The support element 26 is also preferably rigid and linear.

It should be understood that the support element 26 may be integrally formed as one-piece with the intramedullary rod 14, instead of being separable. It should also be understood that, although preferably at right angles with the longitudinal axis of the intramedullary rod 14, a longitudinal extent of the support element 26 may be at a non-perpendicular angle dependent on requirements. Furthermore, although preferably linear, a longitudinal extent of the support element 26 may be at least in part curved in at least one plane, again as necessity dictates.

The femoral body 16 in this embodiment is U-shaped in the plane of the longitudinal axes of the intramedullary rod 14 and the support element 26, providing a front plate 30, a rear plate 32 and a base plate 34 which interconnects the front and rear plates 30, 32. The base plate 34 includes a connector 36, which in this case is a keyway channel integrally formed as part of the base plate 34. The keyway channel 36 is adapted to receive as a sliding fit the support element 26, thereby providing adjustability along the support element 26 for accommodating different kinds of knee size. The femoral body 16 is holdable stationary or substantially stationary relative to the support element 26, and thus the intramedullary rod 14. In particular, the support element 26 prevents or limits any angular displacement of the femoral body 16 relative thereto.

The front and rear plates 30, 32 each includes a head portion 38a, 38b which tapers to a body portion 40a, 40b having a uniform or substantially uniform lateral extent along a majority of its longitudinal extent to the base plate 34. A longitudinal extent of the rear plate 32 is preferably greater than that of the front plate 30, which thereby allows the varus/valgus indicator 22 to be more easily read.

The head portion 38a of the front plate 30 includes a laterally extending slot 42 to slidably receive a locking element 44. The laterally extending slot 42 in this case is curved along its longitudinal extent. The locking element comprises a locking pin 44a having a locking head 44b at its proximal end. A distal end of the locking pin 44a is receivable in a locking aperture 44c of the soft-tissue tensioning connector 20. The laterally extending slot 42 preferably includes teeth or indentations along longitudinal sides thereof. Spacings between the teeth or indentations are complementarily shaped to receive diametrically-opposing locking fins or teeth formed on an underside of the locking head 44b. These spacings beneficially match a scale of the varus/valgus indicator 22. Other locking means can also be considered, for example, the locking pin being screw-threaded to frictionally engage the locking head with the front plate 30.

A longitudinally extending slot 46 is also provided centrally on the rear plate 32, and this extends from the head portion 38b to partway along the body portion 40b.

The soft-tissue tensioning connector 20 is pivotably received in the gap between the front plate 30 and the rear plate 32 of the femoral body 16. A pivot axle 48 extends between the front and rear plates 30, 32 and through the soft-tissue tensioning connector 20, thereby allowing relative angular displacement.

The soft-tissue tensioning connector 20 forms part of a soft-tissue tensioning element 50 of the apparatus 12, which also includes a soft-tissue tensioner 52 to be described hereinafter.

The soft-tissue tensioning connector 20 comprises a waisted plate-like body 54 having a head element 56, body element 58 and foot element 60. The soft-tissue tensioning connector 20 is preferably integrally formed as one-piece. The foot element 60 comprises a centrally located pivot-axle aperture 62, and a soft-tissue tensioner aperture 64 to each side of the pivot-axle aperture 62. The foot element 60 has a width which positions the soft-tissue tensioner apertures 64 outboard of the front plate 30 of the femoral body 16.

The head element 56 of the soft-tissue tensioning connector 20 together with the at least one of the head portions 38a, 38b of the front and rear plates 30, 32 of the femoral body 16 form the varus/valgus indicator 22. A scale 66 is provided, in this case, on the head element 56 of the soft-tissue tensioning connector 20 and a pointer 68 is provided on the upper end of the front plate 30 and the rear plate 32. However, the scale 66 could be provided on one or both of the head portions 38a, 38b of the front and rear plates 30, 32, and one or more pointers 68 could be provided on the head element 56 of the soft-tissue tensioning connector 20.

The head element 56 of the soft-tissue tensioning connector 20 is enlarged relative to its body element 58 in an axial direction of the pivot axle 48. This allows for a pointer slot 70 through the head element 56 in a plane which is parallel to the body element 58 of the soft-tissue tensioning connector 20.

The body element 58 of the soft-tissue tensioning connector 20 is waisted to be accommodated in a body channel 72 of a cutting-jig guide 74. The cutting-jig guide 74 is not required in order to determine an angular alignment of the knee 200 based on the intramedullary rod 14 and the soft-tissue tensioning element 50. However, the cutting-jig guide 74 is extremely beneficial for this invention, allowing a predetermined amount of bone to be resectable from a distal end 206 of the femur 204 based on the most prominent femoral distal condyle 208, as will be understood hereinafter.

A body member 76 of the cutting-jig guide 74 includes the body channel 72 and two outboard cutting-jig locators 78, which in this case are apertures but which may be any suitable locator, such as pins or markers. With the body element 58 of the soft-tissue tensioning connector 20 located in the body channel 72, the cutting-jig guide 74 is slidable longitudinally along and in parallel with the body element 58 of the soft-tissue tensioning connector 20.

Although preferably being a cutting-jig guide 74, so that a cutting jig 80 can be subsequently positioned on a distal end portion 210 of the femur 204, a cutting jig itself may be utilised in place of the cutting-jig guide 74. Such a cutting jig could feature openings, such as slots, for receiving a surgical saw blade to allow the bone resections to be performed without the cutting-jig guide 74.

A body 82 of the condylar locator 18 is also provided in the gap between the front and rear plates 30, 32 of the femoral body 16, pivotably mounted to the pivot axle 48 so as to be interposed between the foot element 60 of the soft-tissue tensioning connector 20 and the rear plate 32 of the femoral body 16. The condylar locator 18 is thus pivotable relative to the femoral body 16 and the soft-tissue tensioning connector 20.

An outrigger 84 extends laterally from each side of the body 82 of the condylar locator 18, and a condylar platform 86 extends in the pivot axis direction from a distal end of each outrigger 84. A condylar abutment surface 88 presented by each condylar platform 86 extends towards the intramedullary rod 14, and the condylar abutment surfaces 88 are coplanar. The condylar abutment surfaces 88 are adapted for seating on their respective medial and lateral distal condyles 208 of the femur 204.

A ligament tension indicator 90 is also provided as part of the condylar locator 18 and the soft-tissue tensioning element 50. The ligament tension indicator 90 includes a pointer arm 92 which extends upwardly from the body 82 of the condylar locator 18 to be movably received in the pointer slot 70 of the head element 56 of the soft-tissue tensioning connector 20. A ligament-tension scale 94 is provided on the head element 56, although it is feasible that the ligament-tension scale 94 could be provided on the pointer arm 92 and an indicator could be provided on the head element 56.

The pointer arm 92 is also connected to the body member 76 of the cutting-jig guide 74. The body member 76 includes a central V-shaped slot 96, and a runner 98 in the form of a pin extends in the direction of the pivot axis P from the pointer arm 92 to be slidably received in the V-shaped slot 96. This arrangement allows the cutting-jig guide 74 or cutting jig if integrally provided to be slidable longitudinally in unison with pivoting movement of the condylar locator 18. In this way, from the pointer arm 92 being at top-dead centre or zero degrees, a distance between a condylar platform 86 and its adjacent outboard cutting-jig locator 78 remains constant as the condylar locator 18 is pivoted and the cutting-jig locator 78 linearly slides.

Referring to FIGS. 3 and 4, a second part 100 of the leg alignment apparatus 12 is shown. This second part 100 is the soft-tissue tensioner 52, engaging with the tibia and the soft-tissue tensioning connector 20 to provide tibio-femoral separation thereby tensioning both medial and lateral collateral ligaments 212.

The soft-tissue tensioner 52 comprises a tibial-platform element 102 and a connector engager 104 which is connected to the tibial-platform element 102 by a rack and pinion 106a, 106b. A tibial platform 108 is provided as part of the tibial-platform element 102, and extends at least substantially in parallel with the connector engager 104. The pinion 106b is drivable by a shaft 110 which is rotatable about its longitudinal extent, thereby driving the rack 106a and thus raising and lowering the connector engager 104.

The connector engager 104 in this embodiment comprises two spaced apart pins 112 which are receivable in the soft-tissue tensioner apertures 64. The pins 112 preferably extend in parallel or substantially parallel with the tibial platform 108.

Although a rack and pinion is utilised, a scissor-jack spreader mechanism, or any other suitable spreader mechanism may be utilised. For example, one or more shims may be used to distract the tibia and femur alone or in combination with the rack and pinion, scissor-jack spreader mechanism, or other spreader mechanism.

The apparatus 12 is preferably formed of biocompatible metal, such as titanium or stainless steel.

Referring to FIGS. 5 and 6, the apparatus 12 is shown applied to the left or right knee joint 214. With the knee 200 in flexion and a bore formed in the intramedullary canal 202, the intramedullary rod 14 is inserted and the support element 26 is attached. The knee joint 214 is then placed in extension, and the femoral body 16 is attached to the support element 26. The rear plate 32 of the femoral body 16 thus lies on or adjacent to the anterior surface of the distal end 206 of the femur 204, and the condylar platform 86 seats on at least the most prominent distal femoral condyle 208.

The tibial platform 108 of the soft-tissue tensioner 52 engages the proximal end of the tibia 216, and the connector engager 104 engages the soft-tissue tensioning connector 20.

To provide a, preferably linear, separation between the tibia 216 and the femur 204, the soft-tissue tensioner 52 is operated to move the connector engager 104 away from the tibial platform 108. This movement causes the condylar abutment surface 88 to be urged against the or each seated condyle 208.

Referring to FIGS. 7a to 7g, 8a to 8g, and 9a to 9g, three methods of determining a distal end resection position for a femur 204 at a knee joint 214 will now be described.

FIGS. 7a to 7g show a neutral leg alignment technique. It is preferable that a pre-operative examination of the knee joint 214 is first conducted, typically by X-ray. This allows preoperative determination of an existing angle between the anatomical axis and the mechanical axis of the femur 204, and whether the leg is in varus, valgus or neutral alignment.

As shown in FIG. 7a and with either the left or right knee 200 in flexion, a proximal end 218 of the tibia 216 is resected perpendicularly or substantially perpendicularly to the mechanical axis of the tibia 216 in a coronal plane and preferably with a three degree posterior slope in the sagittal plane. Although three degrees is suggested, this may be in the range of zero to ten degrees.

The femoral intramedullary canal 202 is also reamed to accept the intramedullary rod 14.

The intramedullary rod 14 is inserted and the knee joint 214 is placed in extension. As shown in FIG. 7b, the support element 26 is connected to the intramedullary rod 14, and the femoral body 16 is engaged with the support element 26. The soft-tissue tensioner 52 is inserted between the tibia 216 and femur 204, so that the tibial platform 108 abuts the resected tibial surface 220, and the connector engager 104 engages the soft-tissue tensioning connector 20.

As shown in FIG. 7c, the soft-tissue tensioner 52 is operated to provide tibio-femoral separation via the condylar locator 18 being forced away from the tibial platform 108. The medial and lateral collateral ligaments 212 and other ligamentous structures surrounding the knee are thus brought into tension.

Due to the pivoting of the condylar locator 18 relative to the soft-tissue tensioning element 50, the varus/valgus indicator 22 of the apparatus 12 indicates an angular alignment of the knee 200 based on the intramedullary rod 14 and/or condylar plane 222 and the distraction between the tibia 216 and the femur 204.

With the ligaments 212 in tension, soft-tissue release is performed until the required neutral alignment, typically being five degrees +/− say three degrees either side, is achieved as indicated by the varus/valgus indicator 22. See FIG. 7d. This deviation of +/− three degrees would typically represent a knee with acceptable constitutional varus/valgus.

As shown in FIG. 7e, the locking element 44 is activated to lock the femoral body 16 relative to the soft-tissue tensioning element 50. This locking holds the cutting-jig guide 74 in place, allowing a cutting tool 114, such as a surgical drill, to be received through the outboard cutting-jig locators 78, as shown in FIG. 7f.

Due to the outboard cutting-jig locator 78 on one side being associated with the condylar platform 86 of the same side and movable in unison therewith whilst maintaining a fixed or substantially fixed separation, a cutting jig location can always be formed at a predetermined distance based on the most prominent distal condyle 208.

With the apparatus 12 then removed, as shown in FIG. 7g, guide pins 116 are inserted at the cutting jig locations and the femoral cutting block or jig 80 is mounted on the distal end 206 of the femur 204. Distal femoral resection 224 can then take place as required.

The apparatus 12 of the present invention is also beneficial, since with the intramedullary rod 14 removed and the knee 200 in flexion, first part 10 of the apparatus 12 can be utilised to set an external rotation of the knee 200 by seating the condylar locator 18 on the medial posterior condyle 226 and using the cutting-jig locator 78 to provide a further cutting jig location in the distal end 206 of the femur 204. In this way, this sets the knee rotation and bone resections based on maintaining the medial collateral ligament isometry.

A further cutting jig can then be mounted on the resected face of the femur, and posterior and anterior resections of the femur can be undertaken, along with respective chamfers.

With the femoral resections completed, the tibial and femoral components of a knee prosthesis can be attached to the respective resected parts, and a meniscal component interposed therebetween in the normal manner.

Turning now to FIGS. 8a to 8g, a constitutional varus/valgus technique is now described. Although described with reference to a varus knee, the same applies to a valgus knee.

For this technique, a pre-operative examination involving X-ray may be dispensed with.

With the knee 200 in flexion, the initial resection of the tibia 216 at right angles to the tibial mechanical axis in the coronal plane and with a three degree posterior slope in the sagittal plane is shown in FIG. 8a, and is similar to that of the neutral leg alignment technique as described above. Although three degrees is suggested, this may be in the range of zero to ten degrees.

With the intramedullary canal 202 reamed and the intramedullary rod 14 inserted, the knee 200 is placed back into extension, the support element 26 is attached, and the femoral body 16 is connected thereto to extend anteriorly over at least a part of the distal end portion 210 of the femur 204. The soft-tissue tensioner 52 is inserted into the meniscal compartment of the joint and connected to the soft-tissue tensioning connector 20, as shown in FIG. 8b.

The soft-tissue tensioner 52 is operated to distract the tibia 216 and femur 204, by the tibial platform 108 seating on the resected tibial surface 220 and the soft-tissue tensioning connector 20 engaging the condylar locator 18. See FIG. 8c.

With the knee 200 distracted, the varus/valgus indicator 22 is read. See FIG. 8d. This reading is based on at least the medial and lateral collateral ligaments 212 being put under tension by the soft-tissue tensioner 52, thereby emulating the patient's normal upright stance.

With the true varus/valgus alignment being determined, a surgeon can then decide whether to perform ligament release to adjust this varus/valgus alignment to a desired value or to leave it as presented. For example, it is often preferable with modern techniques and understanding to maintain a patient's existing varus/valgus alignment to at least some extent, instead of attempting to fully correct the knee 200 to achieve a neutral leg alignment. This may be particularly beneficial for athletes or more active patients. It is now considered that the angle between the anatomical femoral axis and the tibial mechanical axis of between two degrees to eight degrees (being five degrees +/− three degrees) is perfectly acceptable.

Once the varus/valgus alignment has been obtained, the locking element 44 is engaged, as shown in FIG. 8e. This holds the soft-tissue tensioning connector 20 stationary relative to the femoral body 16.

The steps of this technique shown by FIGS. 8f and 8g are the same as those described with reference to FIGS. 7f and 7g above. The engagement of the locking element 44 holds the cutting-jig guide 74 in place, allowing the cutting tool 114, such as a surgical drill, to be received through the outboard cutting-jig locators 78. See FIG. 8f.

The cutting jig location is always formed at a predetermined distance from the distal condyles 208, based on the most prominent distal condyle and due to the synchronous movement of the outboard cutting-jig locator 78 and the respective condylar platform 86. With the apparatus 12 then removed, as shown in FIG. 8g, guide pins 116 are inserted at the cutting jig locations and a femoral cutting block or jig 80 is mounted on the distal end 206 of the femur 204. Distal femoral resection 224 can then take place as required, along with setting external rotation of the knee 200 in the horizontal (transverse) plane, as necessary.

Referring now to FIGS. 9a to 9g, a modified varus/valgus technique is now described. Again, although described in relation to a varus knee 200, the same applies to a valgus knee 200.

This technique is similar to that of the constitutional varus/valgus technique described above with reference to FIGS. 8a to 8g, although a pre-operative examination using X-ray is required to determine an existing angle of the patient's knee 200 between the anatomical and mechanical axes of the femur 204.

As shown in FIG. 9a and with either the left or right knee 200 in flexion, the proximal end 218 of the tibia 216 is resected perpendicularly or substantially perpendicularly to the mechanical axis of the tibia 216 in the coronal plane and with a three degrees slope in the sagittal plane. Although three degrees is suggested, this may be in the range of zero to ten degrees.

The intramedullary canal 202 is also reamed to accept the intramedullary rod 14.

The soft-tissue tensioning connector 20 is then locked via the locking element 44 relative to the femoral body 16, based on an angular alignment of the knee 200 decided during the pre-operative examination, described above. For example, if it is decided that the knee 200 should exhibit five degrees of femoral anatomical valgus, then the varus/valgus indicator 22 is set to five degrees and the locking element 44 is engaged.

The apparatus 12 is then located on the knee 200 as described above with respect to the first and second techniques, see FIG. 9b, and the knee 200 is distracted, as shown in FIG. 9c.

Due to the soft-tissue tensioning connector 20 being held stationary relative to the femoral body 16, as tibio-femoral separation is imparted by the apparatus 12, the condylar locator 18 tilts due to the tensioning of at least the medial and lateral collateral ligaments 212. See FIG. 9d. The pointer arm 92 of the ligament tension indicator 90 indicates a balance of the ligaments on the ligament-tension scale 94 on the head element 56 of the soft-tissue tensioning connector 20. See FIG. 9e. Soft-tissue release is then undertaken, if necessary, until the ligament tension indicator 90 typically reads zero to plus or minus three degrees.

Although not provided in this embodiment, it is feasible that the ligament tension indicator 90 may be lockable relative to the femoral body 16 and/or the soft-tissue tensioning connector 20. This may be provided by the aforementioned locking element 44, for example, by a locking pin being pushable further axially to engage not only the soft-tissue tensioning connector 20 but also the pointer arm 92 of the ligament tension indicator 90. Alternatively, a second separate locking element may be provided.

FIGS. 9f and 9g are similar to FIGS. 7f, 7g and 8f, 8g. The releasable locking holds the cutting-jig guide 74 in place, allowing the cutting tool 114, such as a surgical drill, to be received through the outboard cutting-jig locators 78. With the apparatus 12 then removed, guide pins 116 are inserted at the cutting jig locations and a femoral cutting block or jig 80 is mounted on the distal end 206 of the femur 204. Distal femoral resection 224 then takes place as required.

If required, again the first part 10 of the apparatus 12 can then be utilised to set the external rotation of the knee 200 by seating the condylar locator 18 on the medial posterior condyle 208 and using the cutting-jig locator 78 to provide a further cutting jig location.

With the femoral resections 224 completed, the tibial and femoral components of the knee prosthesis are attached to the respective resected parts, and the meniscal component is interposed therebetween in the normal manner.

A thickness of the prosthetic meniscal component is known, and for example may be 9 mm. By utilising a meniscal scale 118 on the soft-tissue tensioner 52, as shown in FIG. 3, a thickness of the prosthetic meniscal component can be determined following distraction of the knee 200 and prior to distal femoral resection 224. The thickness is preferably from a base of the meniscal component to a lowermost point of its dished condylar seat. The precise positioning of the cutting jig 80 can thus be determined, and can be altered slightly from the cutting-jig guide 74 which is positioned by the movement of the condylar locator 18 and its location on the most prominent, or least worn, distal femoral condyle 208.

Although the soft-tissue tensioner is separate of the soft-tissue tensioning connector, these could be integrally formed together as one-piece, instead of being disengagable.

The ligament tension indicator and associated scale are optional, unless the modified constitutional varus/valgus technique is required.

The soft-tissue tensioning element may be or include shims for direct or indirect engagement with the condylar locator. This may thus dispense with the need for the soft-tissue tensioner, and possibly also the soft-tissue tensioning connector. The shims, which may be considered as spacers, may for example, include pockets for receiving the condylar platforms.

Although the condylar locator is preferably provided by two spaced apart condylar platforms, a single condylar abutting surface which spans between both medial and lateral condyles may be provided.

The apparatus of the present invention is particularly beneficial, as it provides a single apparatus which can be utilised with both left and right knees. Until now, separate devices had to be used for each knee. Furthermore, it is also possible to provide a single apparatus which can be used not only for angular alignment of the femur and tibia of both knees, when the knees are in extension, but also for the setting of external rotation, resection and ligament balancing of the knee utilising a datum taken from the medial posterior femoral condyle.

The embodiments described above are provided by way of examples only, and various other modifications will be apparent to persons skilled in the field without departing from the scope of the invention, as defined by the appended claims.

1. Leg alignment apparatus for use in total arthroplasty of left and right knees, the apparatus comprising an intramedullary rod, a femoral body which locates on or adjacent to an anterior surface of a femur, a support element which extends or is extendable from the femoral body to the intramedullary rod which holds the femoral body stationary or substantially stationary relative to the intramedullary rod, a condylar locator which is pivotable relative to the femoral body and which is seatable on a distal end of the femur, a soft-tissue tensioning element to apply a separation between the femur and the tibia, and a varus/valgus indicator to indicate an angular alignment of the knee based on the intramedullary rod and the soft-tissue tensioning element.

2. Leg alignment apparatus as claimed in claim 1, wherein the soft-tissue tensioning element is able to apply a linear separation between the femur and the tibia.

3. Leg alignment apparatus as claimed in claim 1, wherein the condylar locator is seatable on a distal end of at least one of medial and lateral condyles.

4. (canceled)

5. Leg alignment apparatus as claimed in claim 1, wherein the varus/valgus indicator is able to indicate an angular deviation of the knee from neutral alignment.

6. Leg alignment apparatus as claimed in claim 1, wherein the soft-tissue tensioning element and the femoral body are pivotably interconnected or interconnectable.

7. Leg alignment apparatus as claimed in claim 6, wherein the soft-tissue tensioning element includes a soft-tissue tensioning connector which is pivotably mounted on the femoral body.

8. (canceled)

9. Leg alignment apparatus as claimed in claim 1, wherein the condylar locator includes medial and/or lateral elongate condylar platforms.

10. (canceled)

11. (canceled)

12. Leg alignment apparatus (12) as claimed in claim 1, wherein the soft-tissue tensioning element includes at least one shim element which distracts a tibia and femur.

13. Leg alignment apparatus as claimed in claim 1, wherein the condylar locator includes a ligament tension indicator which is pivotable relative to the soft-tissue tensioning element.

14. (canceled)

15. Leg alignment apparatus as claimed in claim 1, further comprising a cutting-jig guide or a cutting jig which is movable relative to the femoral body and in unison with the condylar locator, so that a predetermined amount of bone is resectable from a distal end of the femur based on the most prominent femoral distal condyle.

16. (canceled)

17. Leg alignment apparatus as claimed in claim 1, wherein the support element is integrally formed as one-piece with the intramedullary rod, and is angularly-offset relative to the longitudinal axis of the intramedullary rod, so as to project in a direction of an anterior surface of a femur.

18. (canceled)

19. (canceled)

20. A method of determining a distal end resection position for a femur at a knee joint, the method comprising the steps of:
a] prior to distal resection and with a leg in extension, providing a separation between a tibia and femur of the knee joint;
b] with the leg still in extension and the separation of step a] applied, determining angular alignment of the knee based on an intramedullary rod extending along a femoral anatomical axis and/or a condylar plane and the separation between the tibia and femur; and
c] determining a position of distal resection from a condylar plane of the femur.

21. The method as claimed in claim 20, further comprising a step d] prior to step a] of resecting a proximal end of the tibia perpendicularly or substantially perpendicularly to the tibial mechanical axis in a coronal plane and in a range of zero to ten degree posterior slope in the sagittal plane.

22. (canceled)

23. (canceled)

24. The method as claimed in claim 20, further comprising a step e] between steps b] and c] of releasing soft tissue spanning between the femur and the tibia to set a required said angular alignment or neutral alignment.

25. The method as claimed in claim 24, wherein, in step e], once the said determined angular alignment or neutral alignment is achieved, the said alignment is locked in place.

26. The method as claimed in claim 25, wherein, prior to step e], the said angular alignment is predetermined and locked in place, and the releasing of soft tissue in step e] causes movement of the condylar plane relative to the tibia.

27. The method as claimed in claim 20, wherein, in step c], the position of the distal femoral resection is set based on the most prominent femoral distal condyle.

28. The method as claimed in claim 20, wherein, in step c], at least one cutting guide locator is formed on an anterior surface of a distal end portion of the femur.

29. The method as claimed in claim 28, wherein the cutting guide locator is a predetermined set distance from a most prominent femoral distal condyle, so that a same predetermined set amount of bone is always resectable from a distal end of the femur.

30. The method as claimed in claim 20, wherein, in step a cutting jig is located on a distal end portion of the femur which guides a cutter during distal resection of the femur.

31. The method as claimed in claim 20, further comprising a step f] subsequent to step c] of placing the leg in flexion, providing a linear separation between the tibia and femur of the knee joint, and further resecting a distal end portion of the femur relative to a medial posterior condyle of the femur so as to set femoral rotation.

32. (canceled)

33. (canceled)

34. (canceled)
